(19) United States
(12) Patent Application Publication
(10) Pub. No.: US 2017/0281176 A1
(43) Pub. Date: Oct. 5, 2017

(54) MEDICAL CLIP DEVICE, METHOD FOR PRODUCING MEDICAL CLIP DEVICE AND METHOD FOR OPERATING THEREOF

(71) Applicant: Kaneka Corporation, Osaka (JP)

(72) Inventors: Naotake Maekubo, Nagano (JP); Masato Tsueda, Nagano (JP)

(73) Assignee: Kaneka Corporation, Osaka (JP)

(21) Appl. No.: 15/626,542

(22) Filed: Jun. 19, 2017

Related U.S. Application Data
(63) Continuation of application No. PCT/JP2015/083788, filed on Dec. 1, 2015.

(30) Foreign Application Priority Data
Dec. 25, 2014 (JP) 2014-263290
Dec. 25, 2014 (JP) 2014-263291

(51) Int. Cl. A61B 17/10; A61B 1/005

(52) U.S. Cl. CPC A61B 17/10; A61B 1/005; A61B 2017/00526

(57) ABSTRACT

A medical clip device includes an outer tubular body; an inner tubular body provided in the outer tubular body; a clip provided at a distal side of the inner tubular body; and a line member including a first end part, a second end part, and a middle part, wherein the first end part and the second end part extend out of the inner tubular body from an inside of the inner tubular body at a proximal side of the inner tubular body, and the middle part is hung on the clip at the distal side of the inner tubular body.

### TECHNICAL FIELD

One or more embodiments of the present invention relate to a medical clip device for excision of a body tissue under an endoscope, a method for producing a medical clip device and a method for operating thereof.

### BACKGROUND

Conventionally, endoscopic submucosal dissection (ESD) and endoscopic mucosal resection (EMR) are adopted for early-stage cancer surgeries with an endoscope on organs such as esophagi or stomachs, which have only a small space for conducting a surgery. In ESD and EMR, some physiological saline solution or hyaluronic acid is infused between the muscular layer and the submucosal layer at a lesion to raise the lesion and surely excise the layers at the lesion. The raised lesion is held and exfoliated with an auxiliary clip with a line member and excised at the bottom with a surgical knife such as an electric scalpel to be removed from the body.

For example, Patent Literature 1 discloses a device for holding a lesion in an endoscopic surgery. The device for an endoscopic surgery includes: a clip having a pair of openable claws facing each other at the tips and connected with each other at the base parts, a fastening ring disposed around the pair of claws to move along the pair of claws for closing the pair of claws, and a connecting board disposed in the fastening ring and having a cut at one end detachably engaged with the base part of the pair of claws and a hole at the other end for engagement; and an extra-fine thread engaged with the base part of the pair of claws and passed through the fastening ring. The clip of the device of Patent Literature 1 for holding a lesion is a commercially available ordinary clip. The extra-fine thread is connected with the base part of such a clip.

### CITATION LIST

### Patent Literature

### Patent Literature 1

Japanese Unexamined Patent Application Publication No. 2007-143869

When the device of Patent Literature 1 is used for excising a lesion in an endoscopic surgery, the operator should carefully take a small clip out of a commercially available ordinary cartridge and tie the line member to the base part of the clip during the surgery. Besides the tying of the line member to the clip prior to a surgery for using the device of Patent Literature 1, the operator should cut the line member with an electric scalpel in the body of a patient for removing the line member from the clip. The cutting of the line member with an electric scalpel involves risks caused by a cutting error. These extra tasks for using the device besides substantial surgical operations take time and extend the surgical time as a whole, which may increase the physical burden on a patient as well as disrupt the operators concentration.

One or more embodiments of the present invention provide a medical clip device that facilitates the connection of a line member with a clip, a method for producing the medical clip device, and a method for operating the medical clip device.

### SUMMARY

A medical clip device of one or more embodiments of the present invention comprises: an outer tubular body; an inner tubular body provided in the outer tubular body; a clip for holding a body tissue, that is provided at a distal side of the inner tubular body; and a line member whose one end part (a first end part) and the other end part (a second end part) extend out of the inner tubular body from an inside of the inner tubular body at a proximal side of the inner tubular body and whose a middle part is hung on the clip at the distal side of the inner tubular body. In the medical clip device, since the middle part of the line member is hung on the clip, the operator can easily connect the line member with the clip or remove the line member from the clip. The medical clip device enables the operator to quickly do the connection and removal the line member with the clip and shorten surgical time, which lessens the physical burden on a patient and helps the operator to stay focused on a surgery.

In the medical clip device, the line member may be coated with a fluororesin. This improves a sliding performance of the line member, the friction between the clip and the line member can be reduced.

In the medical clip device, an inner edge part of the clip hanging the line member may be chamfered. When the line member is pulled, the line member is rubbed mainly against the inner edge part of the clip. Chamfering the inner edge part of the clip prevents the line member from being damaged.

In the medical clip device, a medical fixing member may be anchored to at least one of the one end part and the other end part of the line member. At least one of the one end part and the other end part of the line member thus is anchored, this facilitates the pulling of the line member.

The medical fixing member may comprise: a hole member having a hole part; and a rod member that is engaged with the hole part and holds the line member between the rod member and the hole member, wherein a groove for winding the line member is formed on a surface of the rod member. In the medical fixing member, since the line member is held between the hole member and the rod member, this prevents a displacement of a fixing position of the line member. The line member also can be wound in the side groove in the rod member, a position of the line member thus is surely fixed. Therefore, one or more embodiments of the present invention can be provided the medical clip device in which the operator can steadily pull the line member.

A first slit may be formed on the rod member and a plurality of the grooves may be connected to each other through the first slit. According to one or more embodiments of the present invention, the first slit allows the line member to travel from one groove to the other groove so that the line member passes substantially along the axis of the rod member, which prevents a winding of the line member to accidentally become loose.

A second slit may formed on an outer surface of the hole member, may be connected to the hole part and may extend along the hole part. This allows to insert the line member into the hole part from the second slit in the hole member. The line member can easily be put in the hole part through the second slit in the hole member.

A cut for fixing a part of the line member may be formed on the rod member. A part of the line member can be fixed in the cut in the rod member, which prevents the line member from hindering the operator while puling the line member.

An elastic modulus of the hole member may be higher than that of the rod member. This ensures the engagement of the rod member with the hole member.

The medical clip device may further comprise a first handle connected to a proximal side of the outer tubular body and being movable in an axial direction of the outer tubular body, and a second handle connected to a proximal side of the inner tubular body. Whereby a position in an axis direction of the inner tubular body is fixed by the second handle, the first handle facilitates the moving of the outer tubular body with respect to the inner tubular body in the axial direction.

A projecting part for fixing the line member may be provided on an outer surface of the second handle. The length of the part of the line member extending from the proximal side of the inner tubular body thus can be adjusted depending of the types of surgeries.

In the medical clip device, the clip may have two pinching base plates, and a fastener member may be provided outside of the pinching base plates, has an outer diameter larger than an inner diameter of the inner tubular body, and is movable in the axial direction. When the line member is pulled toward the distal side of the inner tubular body, the proximal end of the fastener member comes into contact with the distal end of the inner tubular body, which prevents the clip from being pulled into the inner tubular body.

A method for producing a medical clip device, in which the medical clip device comprises an outer tubular body, an inner tubular body provided in the outer tubular body, and a clip for holding a body tissue, that is provided at a distal side of the inner tubular body, comprising the steps of: hanging a line member on the clip; and pulling one end part and the other end part of the line member out of the inner tubular body from an inside of the inner tubular body at a proximal side of the inner tubular body. The method for producing a medical clip device of one or more embodiments of the present invention comprises a simple step, which will contribute to mass production of the medical clip devices at low cost. The medical clip device of one or more embodiments of the present invention enables the operator to quickly connect the line member with the clip and shorten surgical time, which lessens the physical burden on a patient and helps the operator to stay focused on a surgery. When using the medical clip device, the operator does not need to touch the clip, which reduces the risk of infection during a surgery.

The method for producing a medical clip device, in which the medical clip device further comprises a medical fixing member including a hole member having a hole part, and a rod member that is engaged with the hole part and holds the line member between the rod member and the hole member, wherein a groove for winding the line member is formed on a surface of the rod member, further comprising the steps of: inserting a part of the line member into the hole part of the hole member; holding the line member between the hole member and the rod member after engaging the rod member to the hole part; and winding the line member around the groove of the rod member. Following these steps, the line member can be easily and surely secured.

A method for operating the medical clip device, in which the medical clip device comprises an outer tubular body, an inner tubular body provided in the outer tubular body, a clip for holding a body tissue, that is provided at a distal side of the inner tubular body, and a line member whose one end part and the other end part extend out of the inner tubular body from an inside of the inner tubular body at a proximal side of the inner tubular body and whose a middle part is hung on the clip at the distal side of the inner tubular body, comprising the steps of: closing the clip; and moving the one end part of the line member to the proximal side. Following these steps, the operator can easily place the clip in a body and withdraw the line member from the body. When the operator needs to use other surgical device, the line member does not hinder the operator from operating the other surgical device.

Further, a method for operating the medical clip device, in which the medical clip device comprises an outer tubular body, an inner tubular body provided in the outer tubular body, a clip for holding a body tissue, that is provided at a distal side of the inner tubular body, and a line member whose one end part and the other end part extend out of the inner tubular body from an inside of the inner tubular body at a proximal side of the inner tubular body and whose a middle part is hung on the clip at the distal side of the inner tubular body, comprising the steps of: closing the clip; and moving the one end part and the other end part of the line member to the proximal side. Following these steps, the operator can easily withdraw the line member together with the clip holding an exfoliated lesion from the body.

A medical clip device comprises a line member whose one end part and the other end part extend out of the inner tubular body from an inside of the inner tubular body at a proximal side of the inner tubular body and whose a middle part is hung on the clip at the distal side of the inner tubular body. The operator thus can easily connect the line member with the clip or remove the line member from the clip. The medical clip device according to one or more embodiments of the present invention enables the operator to quickly do the connection and removal the line member with the clip and shorten surgical time, which lessens the physical burden on a patient and helps the operator to stay focused on a surgery.

A method for producing a medical clip device comprises the steps of: hanging a line member on the clip; and pulling one end part and the other end part of the line member out of the inner tubular body from an inside of the inner tubular body at a proximal side of the inner tubular body. The medical clip device of one or more embodiments of the present invention can be manufactured in a simple way, which will contribute to mass production of the medical clip devices at low cost. The medical clip device enables the operator to quickly connect the line member with the clip and shorten surgical time, which lessens the physical burden on a patient and helps the operator to stay focused on a surgery.

A method for operating the medical clip device comprises the steps of: closing the clip; and moving the one end part of the line member to the proximal side. Following these steps, the operator can easily place the clip in a body and withdraw the line member from the body. When the operator needs to use other surgical device, the line member does not hinder the operator from operating the other surgical device.

A method for operating the medical clip device also comprises the steps of: closing the clip; and moving the one end part and the other end part of the line member to the proximal side. Following these steps, the operator can easily withdraw the line member together with the clip holding an exfoliated lesion from the body.

### BRIEF DESCRIPTION OF THE DRAWINGS

FIG. 1 is a pattern diagram showing a whole structure of a medical clip device of one or more embodiments of the present invention.

FIG. 2 is a side view (including a partial cross-sectional view) of a medical clip device of one or more embodiments of the present invention.

FIGS. 3A and 3B are perspective views of a clip according to one or more embodiments of the present invention.

FIG. 4 is a side view of a clip and a line member according to one or more embodiments of the present invention.

FIG. 5 is a pattern diagram showing a whole structure of a medical clip device of one or more embodiments of the present invention.

FIG. 6 is a side view showing a connection between a medical fixing member, the line member and the clip according to one or more embodiments of the present invention.

FIG. 7 is a side view showing an arrangement between a medical fixing member and the line member according to one or more embodiments of the present invention.

FIG. 8 is a perspective view of a medical fixing member according to one or more embodiments of the present invention.

FIG. 9 is a cross-sectional view (including a partial side view) showing an arrangement between a medical fixing member and the line member.

FIG. 10A is a front view of a hole member according to one or more embodiments of the present invention, FIG. 10B is a cross-sectional view taken along an axial direction of the hole member corresponding to FIG. 10A, and FIG. 10C is a cross-sectional view taken along line X-X of FIG. 10B.

FIG. 11A is a front view of a hole member according to one or more embodiments of the present invention, FIG. 11B is a cross-sectional view taken along an axial direction of the hole member corresponding to FIG. 11A, and FIG. 11C is a cross-sectional view taken along line XI-XI of FIG. 11B.

FIG. 12A is a front view of a hole member according to one or more embodiments of the present invention, FIG. 12B is a cross-sectional view taken along an axial direction of the hole member corresponding to FIG. 12A, and FIG. 12C is a cross-sectional view taken along line XII-XII of FIG. 12B.

FIG. 13A is a front view of a hole member according to one or more embodiments of the present invention, FIG. 13B is a cross-sectional view taken along an axial direction of the hole member corresponding to FIG. 13A, and FIG. 13C is a cross-sectional view taken along line XIII-XIII of FIG. 13B.

FIGS. 14A, 14B, 14C, 14D and 14E are perspective views showing an example of the rod member according to one or more embodiments of the present invention.

FIG. 15 is a side view showing a connection between a medical fixing member, the line member and the clip according to one or more embodiments of the present invention.

FIG. 16 is a cross-sectional view (including a partial side view) of a proximal side of the medical clip device of one or more embodiments of the present invention.

FIG. 17 is a side view of the hole member and the line member according to one or more embodiments of the present invention.

FIG. 18 is a cross-sectional view (including a partial side view) of a medical fixing member according to one or more embodiments of the present invention.

FIG. 19 is a side view of a medical fixing member according to one or more embodiments of the present invention.

FIG. 20 is a side view (including a partial cross-sectional view) showing a method for operating the medical clip device of one or more embodiments of the present invention.

FIG. 21 is a side view (including a partial cross-sectional view) showing a method for operating the medical clip device of one or more embodiments of the present invention.

FIG. 22 is a pattern diagram (including a partial cross-sectional view) showing a method for operating the medical clip device of one or more embodiments of the present invention.

FIG. 23 is a pattern diagram (including a partial cross-sectional view) showing a method for operating the medical clip device of one or more embodiments of the present invention.

FIG. 24 is a pattern diagram showing a method for operating the medical clip device of one or more embodiments of the present invention.

FIG. 25 is a pattern diagram showing a method for operating the medical clip device of one or more embodiments of the present invention.

FIG. 26 is a pattern diagram showing a method for operating the medical clip device of one or more embodiments of the present invention.

FIG. 27 is a pattern diagram showing a method for operating the medical clip device of one or more embodiments of the present invention.

### DESCRIPTION OF THE EMBODIMENTS

Embodiments of the present invention will be specifically explained below, however, the present invention is not restricted by the embodiments described below of course, and can be certainly put into practice after appropriate modifications within in a range meeting the gist of the above and the below, all of which are included in the technical scope of the present invention. In the drawings, hatching, a reference sign for a member may be omitted for convenience, and in such a case, the description and other drawings should be referred to. In addition, sizes of various members in the drawings may differ from the actual sizes thereof, since priority is given to understanding the features of the present invention.

1. Medical Clip Device

A medical clip device of one or more embodiments of the present invention comprises: an outer tubular body; an inner tubular body provided in the outer tubular body; a clip for holding a body tissue, that is provided at a distal side of the inner tubular body; and a line member whose one end part and the other end part extend out of the inner tubular body from an inside of the inner tubular body at a proximal side of the inner tubular body and whose a middle part is hung on the clip at the distal side of the inner tubular body. Therefore, the operator can easily connect the line member with the clip or remove the line member from the clip. The operator can quickly do the connection and removal with the line member and the clip and shorten surgical time, which lessens the physical burden on a patient and helps the operator to stay focused on a surgery.

In early-stage cancer surgeries with an endoscope, a medical clip is generally used for holding a lesion in an organ (countertraction) or for pinching body tissues for hemostasis, suture, or marking. The clip of the medical clip device of one or more embodiments of the present invention is used mainly for holding a target lesion.

The medical clip device controls the opening and closing of the clip to hold or exfoliate a lesion or to remove a lesion from the body. In this description, the medical clip device may simply be referred to as a “clip device.”

In the present disclosure, unless otherwise specified, an axial direction refers to a long axis direction of the inner tubular body, and a proximal side in the axial direction refers to a direction of an operator's hand side, while a distal side refers to a direction opposite to the proximal side.

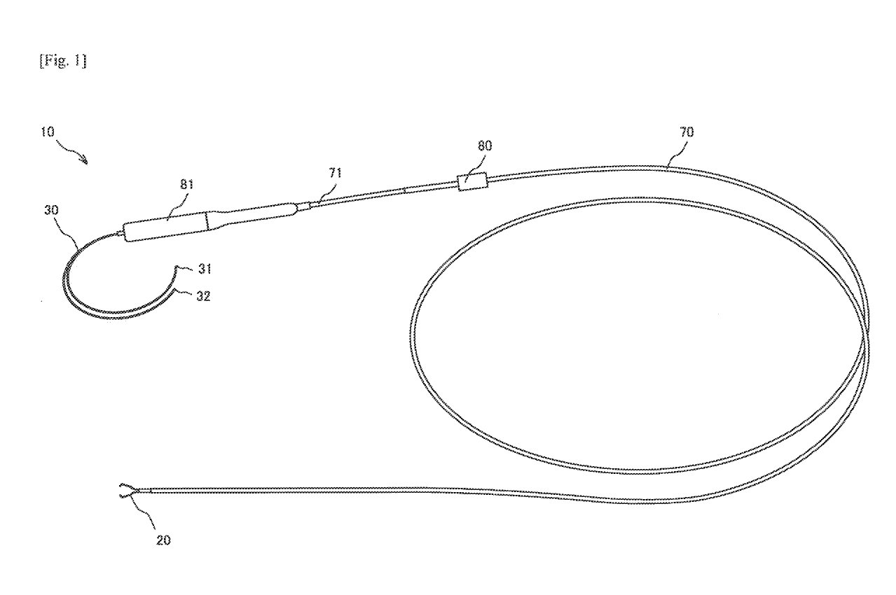

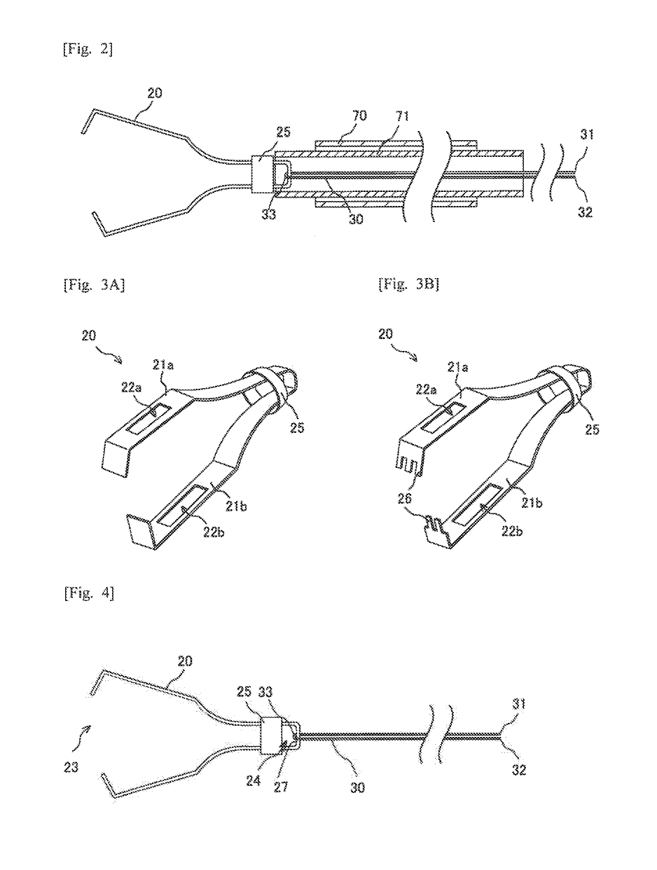

FIG. 1 is a pattern diagram showing a whole structure of a medical clip device 10 of one or more embodiments of the present invention, and FIG. 2 is a side view (including a partial cross-sectional view) of a medical clip device 10 of one or more embodiments of the present invention. The clip device 10 comprises an outer tubular body 70, an inner tubular body 71, a clip 20 and a line member 30.

The outer tubular body 70 places the inner tubular body 71 in the lumen thereof. The outer tubular body 70 protects a forceps opening of an endoscope, the inner wall of a forceps channel and body tissues other than a target tissue from being damaged by the clip 20 while the clip 20 is being sent from the forceps opening of an endoscope through the forceps channel to the target tissue in a patient.

The outer tubular body 70 may be made of synthetic resins such as polytetrafluoroethylene (PTFE) or polypropylene so that the outer tubular body 70 can bend more flexibly along the movement of a curved tube of an endoscope. The outer tubular body 70 may be made of transparent or translucent materials so that the operator can see the position of the inner tubular body 71 in the outer tubular body 70.

The inner tubular body 71 places a part of the line member 30 in the lumen thereof. The inner tubular body allows to adjust an opening/a closing of the clip 20 by being moved with respect to the clip 20 back and forth in the axial direction. The inner tubular body 71 may have a balanced combination of flexibility to bend along the shape of a body cavity and rigidity to reach a target tissue.

The inner tubular body 71 may be made of a coiled metal member, a plurality of short cylindrical joint pieces rotatably connected in the axial direction, or a synthetic resin such as polytetrafluoroethylene (PTFE), and may be made of a high-strength metal coil.

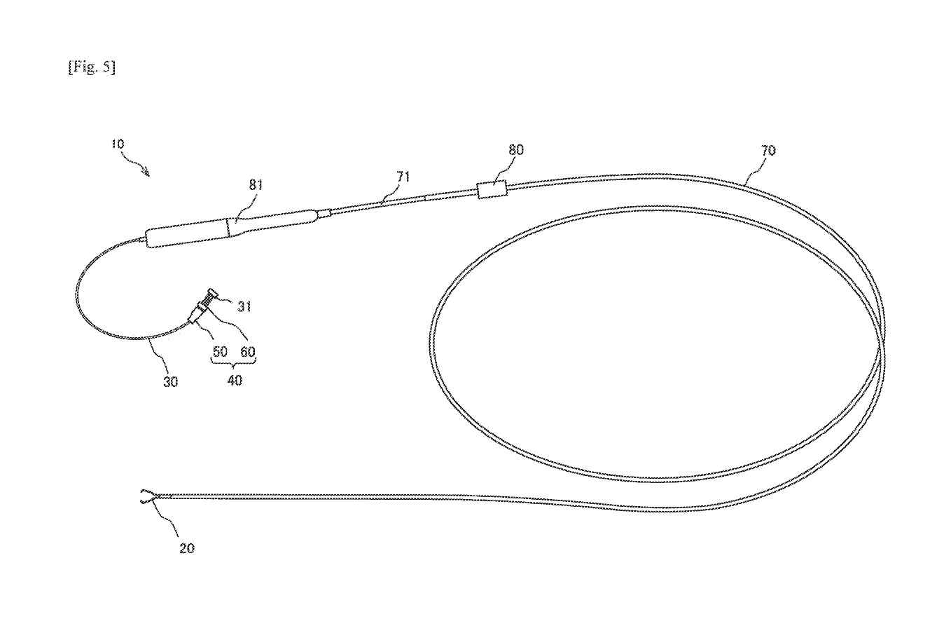

FIGS. 3A and 3B are perspective views of the clip 20 according to one or more embodiments of the present invention. The clip 20 according to one or more embodiments of the present invention may include two pinching base plates 21a and 21b. The clip 20 of FIGS. 3A and 3B includes the pinching base plates 21a and 21b. The pinching base plates 21a and 21b are opened or closed to hold target tissues such as a lesion. The pinching base plates 21a and 21b may have openings 22a and 22b, respectively, at the distal sides so that the clip 20 can easily deform.

As shown in FIGS. 3A and 3B, the pinching base plates 21a and 21b of the clip 20 may have greater widths at the distal sides than at the proximal sides. As shown in FIGS. 3A and 3B, one of the pinching base plate of the clip 20 or the pinching base plate 21a may be longer than the other pinching base plate or the pinching base plate 21b at the distal sides in the axial direction for ensuring the holding of a lesion by the clip 20. As shown in FIG. 3B, the clip 20 may have indentations 26 at the distal ends for ensuring the holding of a lesion by the clip 20.

The clip 20 is provided with a fastener member 25 whose an outer diameter is larger than the inner diameter of the inner tubular body 71 and is movable in the axial direction. Specifically, the fastener member 25 may have a circular cylindrical shape, for example, and is disposed around the V-shaped clip 20 at the proximal side. A pressure to be applied from outside of the pinching base plates 21a and 21b inward thereof is changed by being moved the fastener member 25 along two pinching base plates 21a and 21b, which allows the clip 20 to open and close. The fastener member 25 may have a circular cylindrical shape or a polygonal cylindrical shape. The fastener member 25 has an outer diameter larger than the inner diameter of the inner tubular body 71. When the line member 30 is pulled toward the proximal side, the proximal end of the fastener member 25 comes into contact with the distal end of the inner tubular body 71, which prevents the clip 20 from being pulled into the inner tubular body 71.

The line member 30 is a traction member hung on the clip 20 for holding a lesion (specifically at a given area of the pinching base plates 21a and 21b except for the fastener member 25). The line member 30 is used for removing the clip 20 and a lesion from the body. The line member 30 includes one end part 31, the other end part 32, and a middle part 33 between the one end part 31 and the other end part 32. The one end part 31 accounts for 30% of the line member 30, the other end part 32 accounts for 30% of the line member 30, and the middle part 33, which is the rest of the line member 30 other than the one end part 31 and the other end part 32, accounts for 40% of the line member 30.

In the line member 30 according to one or more embodiments of the present invention, the one end part 31 and the other end part 32 extend out of the inner tubular body 71 from an inside of the inner tubular body 71 at a proximal side of the inner tubular body 71. As shown in FIGS. 1 and 2, the one end part 31 and the other end part 32 are exposed out of the inner tubular body 71 at the proximal side of the inner tubular body 71, that is, at the side opposite to the clip 20.

In one or more embodiments of the present invention, the middle part 33 of the line member 30 is hung on the clip 20 at the distal side of the inner tubular body 71. The inner tubular body 71 and the clip 20 are arranged so that the distal side of the inner tubular body 71 faces the proximal side of the clip 20.

FIG. 4 is a side view of the clip 20 and the line member 30 according to one or more embodiments of the present invention, showing the arrangement of the clip 20 and the line member 30. As shown in FIG. 4, the middle part 33 of the line member 30 may be hung on the clip 20 at the U-shaped proximal part formed by the pinching base plates 21a and 21b of the clip 20. The middle part 33 of the line member 30 may be hung on the clip 20 at the openings 22a and 22b in the pinching base plates 21a and 21b, respectively, or at a through-hole (not shown) formed on the proximal side of the clip 20.

Actually, the middle part 33 of the line member 30 is simply hung on the clip 20, so that the line member 30 can be removed from the clip 20 only by pulling one of the one end part 31 and the other end part 32 of the line member 30. As described above, in the clip device 10 of one or more embodiments of the present invention, the line member 30 can easily be connected with the clip 20 and removed from the clip 20.

The line member 30 may be made of a single yarn or a twisted thread containing plural threads. The line member 30 may be made by weaving a plurality of single yarns or twisted threads.

The line member 30 may be composed of a material having biocompatibility and strength, may be composed of polyamide resin such as nylon, polyolefin resin such as polypropylene, polyester resin such as polyethylene terephthalate, and fluorine resin such as PTFE.

To smoothly withdraw the line member 30 from a body, the line member 30 may have an appropriate length depending on the types of surgeries. Therefore, the length of the line member 30 may be 2.2 times or more of that of the inner tubular body 71, 2.4 times or more and 2.6 times or more.

The maximum length of the line member 30 is not particularly limited, to prevent operating of the line member 30, the length of the line member 30 may be 3.4 times or less, 3.2 times or less and 3.0 times or less.

Large friction between the line member 30 and the clip 20 may break the line member 30 when the operator holds and pulls the one end part 31 of the line member 30 to withdraw the line member 30 from the body. The line member 30 thus may be coated with a fluororesin to improve its sliding performance and reduce the friction with the clip 20. When the line member 30 is hollow, the line member 30 may be coated with a fluororesin on its outer surface.

The fluororesin can be PTFE, tetrafluoroethylene-hexafluoropropylene copolymer (FEP), ethylene-tetrafluoroethylene copolymer (ETFE).

The line member 30 may be coated with a fluororesin in a known method such as immersion coating, spray coating, fluidized-bed coating, or kneader coating.

An inner edge part 27 of the clip 20 hanging the line member 30 may be chamfered. When the line member 30 is pulled, the line member 30 is rubbed mainly against the inner edge part 27 of the clip 20. Chamfering the inner edge part 27 of the clip 20 prevents the line member 30 from being damaged. It may be desired to chamfer the inner edge part 27 at the proximal side of the clip 20. The inner edge part 27 at the proximal side of the clip 20 is the inner edge area of one third of the clip 20 on the most proximal side in the longitudinal direction of the clip 20 when viewed from the above, for example. The inner edge part 27 of the clip 20 may be chamfered by a known method such as round chamfering with a chamfering machine.

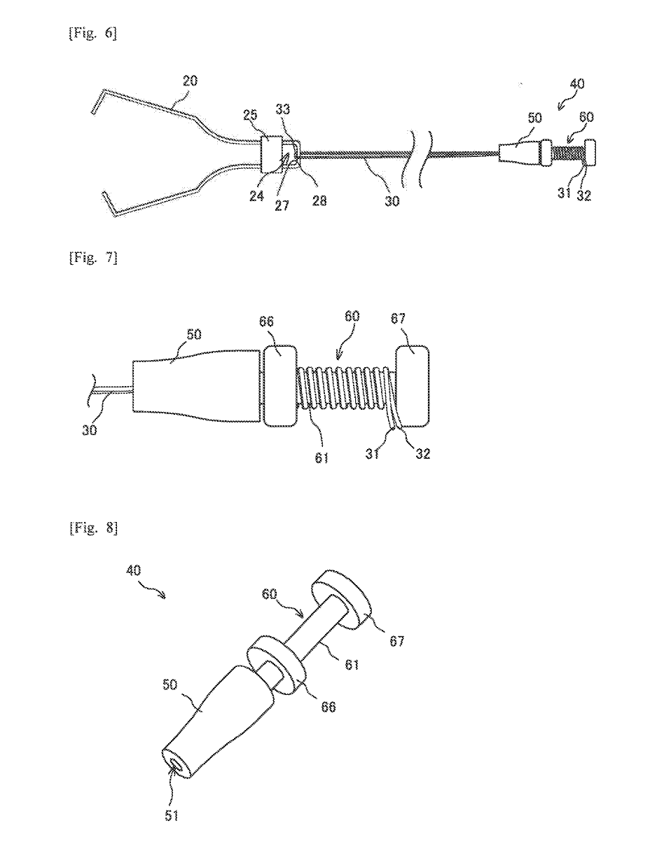

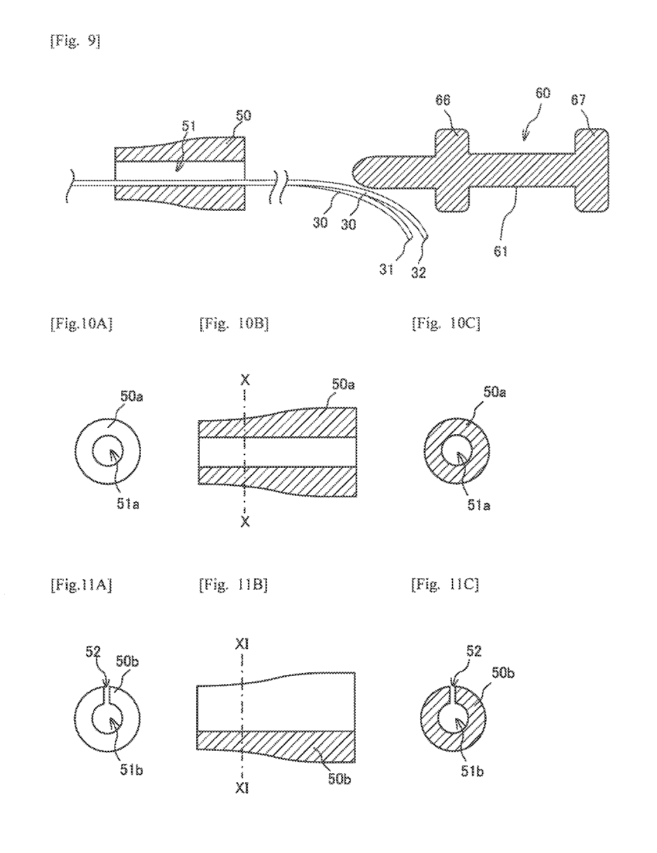

FIG. 5 is a pattern diagram showing a whole structure of a medical clip device 10 anchored a medical fixing member 40 according to one or more embodiments of the present invention. FIG. 6 is a side view showing a connection between a medical fixing member 40, the line member 30 and the clip 20 according to one or more embodiments of the present invention. FIG. 7 is a side view showing an arrangement between a medical fixing member 40 and the line member 30. FIG. 8 is a perspective view of a medical fixing member 40 according to one or more embodiments of the present invention. FIG. 9 is a cross-sectional view (including a partial side view) showing an arrangement between a medical fixing member 40 and the line member 30.

As shown in FIGS. 7 to 9, a medical fixing member 40 may be anchored to at least one of the one end part 31 and the other end part 32 of the line member 30. The medical fixing member 40 is used for anchoring at least one of the one end part 31 and the other end part 32 of the line member 30. The medical fixing member 40 may be held by the operator for facilitating the pulling of the line member 30 by the operator. In the following example, both of the one end part and the other end part of the line member 30 are secured to the medical fixing member 40, however, other embodiments are possible.

As shown in FIGS. 8 and 9, the medical fixing member 40 may comprise a hole member 50 having a hole part 51, and a rod member 60 that is engaged with the hole part 51 and holds the line member 30 between the rod member 60 and the hole member 50, wherein a groove 61 for winding the line member 30 is formed on a surface of the rod member 60.

The hole member 50 has a hole part 51 into which the line member 30 is inserted. The hole member 50 with the hole part 51 may be a cylindrical member with a through-hole formed in the axial direction of the rod member 60 corresponding to the longitudinal direction of the hole member 50. The hole member 50 may be a cylindrical member with a closed bottom. In this case, the hole part 51 is a blind hole formed in the axial direction of the rod member 60.

In the case of the medical fixing member 40 of which the cylindrical hole member 50 has the longitudinal through-hole part 51, the line member 30 is held between the hole member 50 and the rod member 60 so that the position of the line member 30 is fixed with respect to the clip 20. The line member 30 may be wound in the groove 61 in the rod member 60 for firm securing. With the medical clip device 10 according to one or more embodiments of the present invention, the operator can steadily pull the line member 30 by pulling the medical fixing member 40.

The hole part 51 is larger than the outer diameter of the line member 30. When the hole member 50 has an elastic modulus higher than that of the rod member 60, the hole part 51 may be smaller than the outer diameter of the rod member 60 before accepting the line member 30 and the rod member 60.

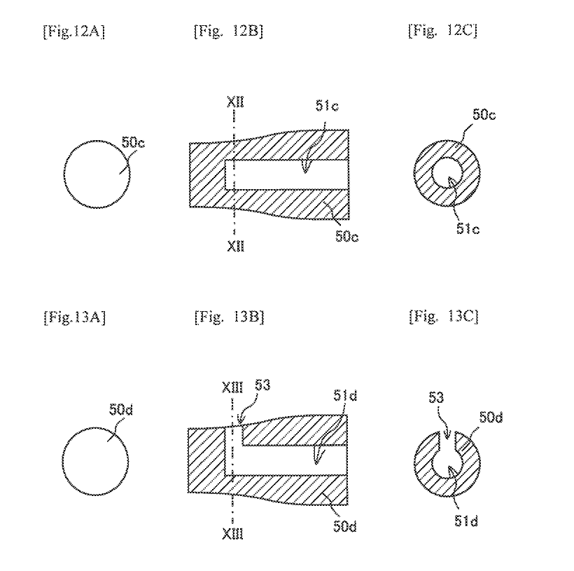

An example of a constitution of the hole member will be described with reference to FIGS. 10 to 13. FIGS. 10A, 11A, 12A and 13A are front views of a hole member according to one or more embodiments of the present invention. Each of FIGS. 10B, 11B, 12B and 13B is a cross-sectional view taken along the axial direction of the hole member corresponding to each of FIGS. 10A, 11A, 12A and 13A. Each of FIGS. 10C, 11C, 12C and 13C is cross-sectional view taken along each of line X-X of FIG. 10B, line XI-XI of FIG. 11B, line XII-XII of FIG. 12B and line XIII-XIII of FIG. 13B.

As shown in FIGS. 10A to 10C, the hole part 51a may pierce the hole member 50a in the axial direction of the rod member 60. In this structure, the rod member 60 aligns with the line member 30 in the hole part 51a in the axial direction, which enables the operator to steadily pull the line member 30.

As shown in FIGS. 11A to 11C, a second slit 52 is formed on an outer surface of the hole member 50b, is connected to the hole part 51b and extends along the hole part 51b. The line member 30 can easily be put in the hole part 51b through the second slit 52 in the hole member 50b.

As shown in FIGS. 12A to 12C, the hole member 50c may have a blind hole part 51c extending along the axial direction of the rod member 60. The hole part 51c formed in this manner is suitable for the case in which the hole member 50c is disposed proximal to the rod member 60. In this case, the length of the line member 30 between a medical clip or the like and the medical fixing member 40 can be adjusted by changing the number of the winding of the line member 30 in the groove 61 in the rod member 60.

As shown in FIGS. 13A to 13C, the hole member 50d may have a blind hole part 51d extending along the axial direction of the rod member 60 and an opening 53 formed in its side. The opening 53 is in communication with the hole part 51d. The hole part 51d formed in this manner is suitable for the case in which the hole member 50d is disposed proximal to the rod member 60. In this case, the length of the line member 30 between a medical clip or the like and the medical fixing member 40 can be adjusted by changing the number of the winding of the line member 30 in the groove 61 in the rod member 60.

The rod member 60 is engaged with the hole part 51 in the hole member 50 so that the line member 30 is held between the hole member 50 and the rod member 60. The rod member 60 may be held by the operator for pulling the line member 30.

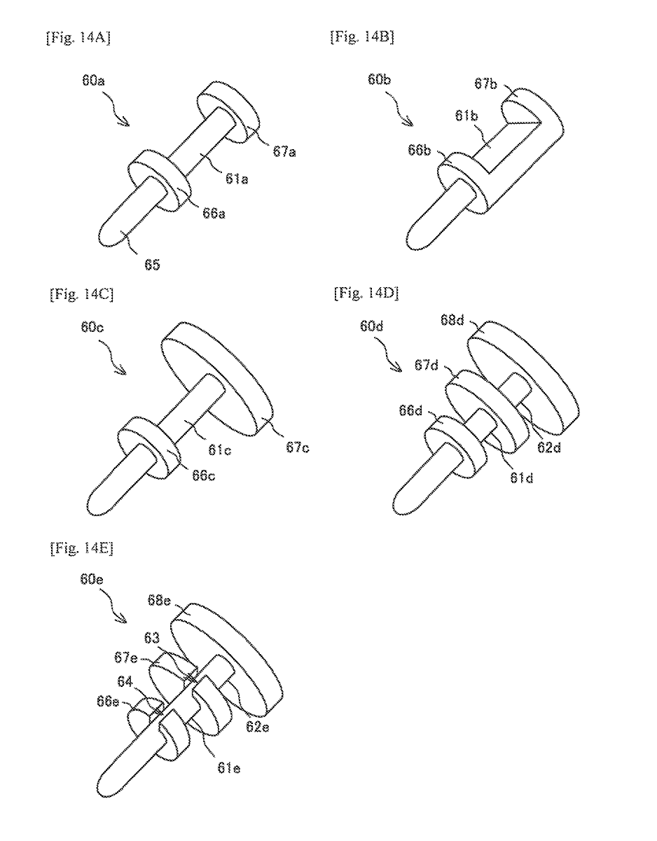

An example of a constitution of the rod member will be described with reference to FIG. 14. FIGS. 14A to 14E are perspective views showing an example of the rod member 60 according to the present invention. As shown in FIG. 14A, the rod member 60a may have a tapered part 65. When the rod member 60a is engaged with the hole member 50, the tapered part 65 facilitates the insertion of the rod member 60a into the hole part 51 in the hole member 50.

A groove 61 for winding the line member 30 is formed on a surface of the rod member 60. In the case of the medical fixing member 40 of which the cylindrical hole member 50 has the longitudinal through-hole part 51, the line member 30 is held between the hole member 50 and the rod member 60 for securing. In addition, the line member 30 can be wound in the side groove 61 in the rod member 60 for firm securing. The operator thus can steadily pull the line member 30.

In one or more embodiments of the present invention, the groove 61 means any depressed area with any depth or width. The rod member 60 may have one groove 61 or more around the entire circumference or a part of the circumference.

In one or more embodiments of the present invention, the line member 30 should be wound in the groove 61 in the rod member 60 one and a half turns or more, for example, two turns or more, and three turns or more.

The rod member 60 with the groove 61 will have a higher area than the groove 61 in the radial direction. The rod member 60 may have collars 66 and 67 higher than the groove 61 in the radial direction. In the rod member 60, the collars 66 and 67 function as walls for the line member 30 wound in the groove 61. The collars may have any radial heights or widths. The rod member 60 may have one collar or more around the entire circumference or a part of the circumference.

As shown in FIG. 14A, the rod member 60a may have the groove 61a around the entire circumference. Even if the line member 30 is wound on the rod member 60a many turns, the groove 61a helps the operator to hold the rod member 60a. FIG. 14A shows the example in which the rod member 60a has the collars 66a and 67a with substantially equal diameters.

As shown in FIG. 14B, the rod member 60b may have the groove 61b formed around a part of the circumference. The rod member 60b can easily be engaged with the hole member 50 by holding a part of the rod member 60b other than the groove 61b. FIG. 14B shows the example in which the rod member 60b has collars 66b and 67b around a part of the circumference.

As shown in FIG. 14C, the rod member 60c may have a larger outer diameter at the side opposite to the side to be inserted into the hole part 51 in the hole member 50. With this structure, the rod member 60c can easily be inserted into the hole part 51 in the hole member 50 by holding the rod member 60c when the rod member 60c is engaged with the hole member 50. FIG. 14C shows the example in which the rod member 60c includes collars 66c and 67c, and the outer diameter of the collar 67c is larger than that of the collar 66c.

As shown in FIG. 14D, the rod member 60d may have a plurality of grooves (such as two grooves 61d and 62d in FIG. 14D). The line member 30 can be wound in each of the grooves 61d and 62d for firm securing. FIG. 14D shows the example in which the rod member 60d has collars 66d, 67d, and 68d. In the rod member 60d of FIG. 14D, the outer diameters of the collars increase with distance from the tapered part 65, that is, the collar 67d is larger than the collar 66d and the collar 68d is larger than the collar 67d.

As shown in FIG. 14E, the rod member 60e may have a first slit 63 for connecting the grooves 61e and 62e. The first slit 63 allows the line member 30 to travel from the groove 61e to the groove 62e, for example, so that the line member 30 passes substantially along the axis of the rod member 60e, which prevents a winding of the line member 30 to accidentally become loose. In the rod member 60e of FIG. 14E, the outer diameters of the collars increase with distance from the tapered part 65, that is, the collar 67e is larger than the collar 66e and the collar 68e is larger than the collar 67e.

As shown in FIG. 14E, the rod member 60e may have another first slit 64 for allowing the line member 30 between the rod member 60e and the hole member 50 to travel from the hole member 50 to the groove 61e so that the line member 30 passes substantially along the axis of the rod member 60e.

The groove 61 in the rod member 60 may be formed in a spiral (not shown). The line member 30 is wound in the spiral groove 61 for firm securing.

A cut for fixing a part of the line member 30 may be formed on the rod member 60. A part of the line member 30 can be fixed in the cut in the rod member 60, which prevents the line member 30 from hindering the operator while puling the line member 30.

The hole member 50 and the rod member 60 can be composed of a polymer material such as polyethylene, polypropylene and polyethylene terephthalate.

An elastic modulus of the hole member 50 may be higher than that of the rod member 60. This ensures the engagement of the rod member 60 with the hole member 50.

The rod member 60 may be disposed proximal to the hole member 50. In this case, the line member 30 is held between the hole member 50 and the rod member 60 and then wound in the groove 61 in the rod member 60 so that the length of the line member 30 between a medical clip or the like and the medical fixing member 40 can be fixed.

The hole member 50 may be disposed proximal to the rod member 60. In this case, the line member 30 is wound in the groove 61 in the rod member 60 and then held between the hole member 50 and the rod member 60 so that the length of the line member 30 between a medical clip or the like and the medical fixing member 40 can be adjusted by changing the number of the winding of the line member 30 in the groove 61 in the rod member 60.

The outer diameter of the medical fixing member 40 may be larger than the inner diameter of the inner tubular body 71. This allows the medical fixing member 40 to be caught on the proximal end of the inner tubular body 71, which prevents the one end part 31 and the other end part 32 of the line member 30 from entering the inner tubular body 71 through the proximal side of the inner tubular body 71.

FIG. 6 shows the example in which both of the one end part and the other end part of the line member 30 are secured to the medical fixing member 40, however, the line member 30 may be connected with the medical fixing member 40 of the clip device according to one or more embodiments of the present invention in the following manner.

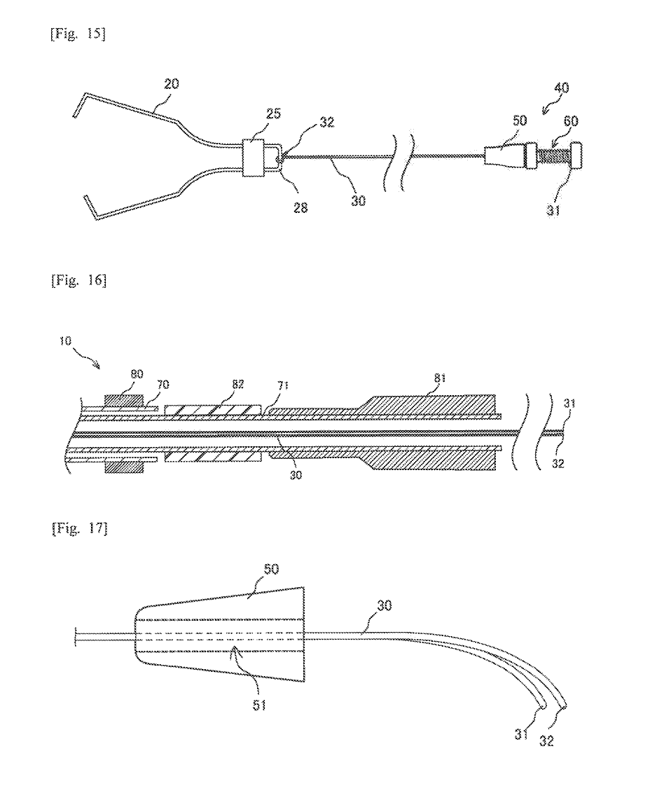

FIG. 15 is a side view showing the other example of an arrangement between the medical fixing member 40 and the line member 30 according to one or more embodiments of the present invention. As shown in FIG. 15, a medical fixing member 40 may be anchored to the one end part 31 or the other end part 32 of the line member 30. This is suitable for connecting one of the one end part 31 and the other end part 32 of the line member 30 with a medical clip or the like.

The line member 30 may be anchored to the medical fixing member 40 at the middle part 33 (not shown). This is suitable for connecting both of the one end part 31 and the other end part 32 of the line member 30 with a medical clip or the like. The line member 30 may be anchored to the medical fixing member 40 at the one end part 31 and the middle part 33, at the part between the one end part 31 and the middle part 33, or at the part between the other end part 32 and the middle part 33.

FIG. 16 is a cross-sectional view (including a partial side view) of a proximal side of the medical clip device 10 according to an embodiment of one or more embodiments of the present invention. One or more embodiments of the present invention also include the clip device 10 comprising a first handle 80 connected to a proximal side of the outer tubular body 70 and being movable in an axial direction of the outer tubular body 70 and a second handle 81 connected to a proximal side of the inner tubular body 71.

As shown in FIGS. 1, 5 and 16, the first handle 80 is connected to the proximal side of the outer tubular body 70. The first handle 80 on the clip device 10 facilitates the moving of the outer tubular body 70 in the axial direction.

The distal sides of the outer tubular body 70 and the inner tubular body 71 are inserted into a body through the forceps channel of an endoscope. The outer diameter of the first handle 80 may be larger than the inner diameter of the forceps channel of an endoscope. The outer diameter of the second handle 81 may be larger than the inner diameter of the forceps channel of an endoscope. The first handle 80 and the second handle 81 prevent the outer tubular body 70 and the inner tubular body 71, respectively, from completely entering the forceps channel of an endoscope, otherwise the outer tubular body 70 would become substantially unable to be moved in the axial direction.

The first handle 80 may be connected with the outer tubular body 70 by mechanical securing such as fitting, using screws, or caulking, bonding using adhesives such as polyurethane adhesives, epoxy adhesives, cyano adhesives, or silicone adhesives, or welding.

In FIGS. 1, 5, and 16, the second handle 81 is connected with the inner tubular body 71 at the proximal side. The second handle 81 on the clip device 10 fixes the position of the inner tubular body 71 in the axial direction, which facilitates the moving of the outer tubular body 70 with respect to the inner tubular body 71 in the axial direction.

Except when being pulled by the operator, the part of the line member 30 extending from the proximal side of the inner tubular body 71 may tangle with other surgical device than the clip device 10 and hinder the operator from smoothly conducting a surgery. A projecting part for fixing the line member 30 may be provided on an outer surface of the second handle 81. The part of the line member 30 extending from the proximal side of the inner tubular body 71 can be hung on the projecting part so that the length of the part of the line member 30 extending from the proximal side of the inner tubular body 71 can be adjusted depending of the types of surgeries. In this example, the second handle 81 has a projecting part on the outer surface for fixing the line member 30, however, the second handle 81 may have a groove on the outer surface for fixing the line member 30.

Similar to the connection between the first handle 80 and the outer tubular body 70, the second handle 81 may be connected with the inner tubular body 71 by mechanical securing such as fitting, using screws, or caulking, bonding using adhesives such as polyurethane adhesives, epoxy adhesives, cyano adhesives, or silicone adhesives, or welding.

The clip 20 is disposed in the outer tubular body 70, and then the clip device 10 is inserted into the forceps channel of an endoscope, which prevents the clip 20 from scratching the inner wall of the forceps channel.

The outer tubular body 70, of which the outer surface is in contact with the inner wall of the forceps channel, may accidentally move in the axial direction, which may cause the clip to come out of the outer tubular body 70 in the forceps channel. To prevent this, the clip device 10 may have a spacer 82 disposed on the outer surface of the inner tubular body 71 proximally to the outer tubular body 70 and distally to the second handle 81. The proximal end of the spacer 82 comes into contact with the distal end of the second handle 81 and the distal end of the spacer 82 comes into contact with the proximal end of the outer tubular body 70, which prevents the outer tubular body 70 from excessively moving in the axial direction so that the clip 20 does not come out of the outer tubular body 70 in the forceps channel.

The spacer 82 may be a circular hollow cylinder or a rectangular hollow cylinder, or a board member having an opening or a cut. The outer diameter of the spacer 82 at the distal end may be larger than the inner diameter of the outer tubular body 70 at the proximal end to prevent the spacer 82 from entering the outer tubular body 70. The inner diameter of the spacer 82 at the distal end is smaller than the outer diameter of the outer tubular body 70 at the proximal end so that the distal end of the spacer 82 comes into contact with the proximal end of the outer tubular body 70. The inner diameter of the spacer 82 at the proximal end is smaller than the outer diameter of the second handle 81 at the distal end to prevent the second handle 81 from entering the spacer 82, otherwise the outer tubular body 70 would accidentally move in the axial direction.

The spacer 82 may have a slit in communication with a lumen thereof and extending along the axial direction, or an engaging mechanism such as a hook-and-loop fastener or a snap fastener to facilitate the attachment to the inner tubular body 71 or the removal from the inner tubular body 71. The spacer 82 may be made of a synthetic resin such as a polypropylene, or a foamed plastic such as a polyurethane foam.

2. Method for Producing Medical Clip Device

One or more embodiments of the present invention provide a method for producing a medical clip device, in which the medical clip device comprises an outer tubular body, an inner tubular body provided in the outer tubular body, and a clip for holding a body tissue, that is provided at a distal side of the inner tubular body, comprising the steps of: hanging a line member on the clip; and pulling one end part and the other end part of the line member out of the inner tubular body from an inside of the inner tubular body at a proximal side of the inner tubular body.

The method for producing the medical clip device 10 of one or more embodiments of the present invention will now be described in detail. The medical clip device 10 includes the constituent components described in the above section “1. Medical Clip Device.”

First, the outer tubular body 70, the inner tubular body 71, the clip 20, and the line member 30 are prepared for producing the clip device 10 (step S11). The fastener member 25 may be disposed around the pinching base plates 21a and 21b of the clip 20 to move in the axial direction. The fastener member 25 has an outer diameter larger than the inner diameter of the inner tubular body 71.

The line member 30 is then passed between the pinching base plates 21a and 21b of the clip 20 (or through an opening 23 in FIG. 4, for example) to be hung on the clip 20 at the proximal side of the clip 20 or the distal side of the inner tubular body 71 (step S12).

The one end part 31 and the other end part 32 of the line member 30 are then inserted into the fastener member 25 to dispose the fastener member 25 around the pinching base plates 21a and 21b of the clip 20 with the line member 30 (step S13). When the fastener member 25 is not used for the clip 20, this step should be omitted.

When the fastener member 25 is used for the clip 20, the above steps S12 and S13 may be replaced with a step of inserting the one end part 31 of the line member 30 between the clip 20 and the fastener 25 (or through an opening 24 in FIG. 4, for example) to hang the line member 30 on the clip 20 (step S14).

The one end part 31 and the other end part 32 of the line member 30 are then inserted into the inner tubular body 71 through the distal side of the inner tubular body 71 (step S15).

The one end part 31 and the other end part 32 of the line member 30 are pulled out of the inner tubular body 71 from an inside of the inner tubular body 71 at a proximal side of the inner tubular body 71 (step S16), while the line member 30 is hung on the clip 20 at the distal side of the inner tubular body 71.

One end part of the inner tubular body 71 is then inserted into the outer tubular body 70 to dispose the inner tubular body 71 in the outer tubular body 70 (step S17).

To enable the operator to accurately and easily pull the line member 30, the middle part 33 of the line member 30 is determined so that the length of the line member 30 between the one end part 31 and the middle part 33 is substantially equal to the length of the line member 30 between the other end part 32 and the middle part 33 (step S18). Step 18 may be performed before step S17.

The one end part 31 and the other end part 32 of the line member 30 are then connected to the medical fixing member 40 (step S19). The medical fixing member 40 includes the hole member 50 having a hole part 51, and a rod member 60 that is engaged with the hole part 51 and holds the line member 30 between the rod member 60 and the hole member 50, wherein a groove 61 for winding the line member 30 is formed on a surface of the rod member 60. This step may be omitted.

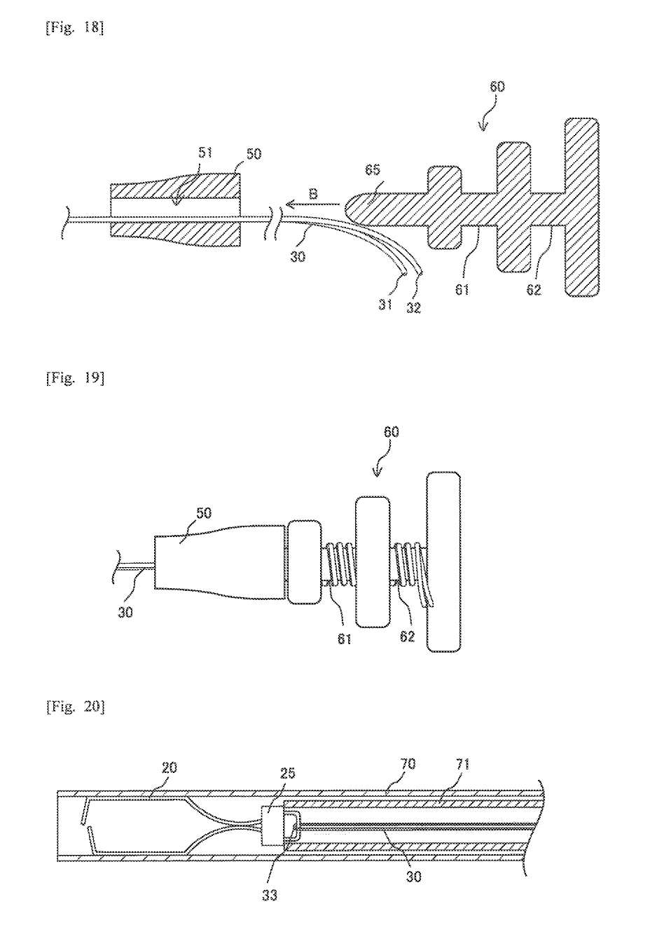

A method for assembling the medical fixing member 40 of one or more embodiments of the present invention will now be described in detail with reference to FIGS. 17 to 19. FIG. 17 is a side view of the hole member 50 and the line member 30 according to one or more embodiments of the present invention. FIG. 18 is a cross-sectional view (including a partial side view) of the medical fixing member 40 according to one or more embodiments of the present invention. FIG. 19 is a side view of the medical fixing member 40 according to one or more embodiments of the present invention. The medical fixing member 40 includes the constituent components described in the above section “1. Medical Clip Device.”

First, the hole member 50, the rod member 60, and the line member 30 are prepared for assembling the medical fixing member 40 (step S19-1).

A part of the line member 30 is then inserted into the hole part 51 of the hole member 50 (step S19-2). When the hole member 50 has the second slit 52, the line member 30 may be put in the hole part 51 through the second slit 52. In FIG. 17, the one end part 31 and the other end part 32 of the line member 30 are disposed in the hole part 51, however, the number of the one end part and the other end part of the line member 30 disposed in the hole part 51 is not limited.

The line member 30 is held between the hole member 50 and the rod member 60 after engaging the rod member 60 to the hole part 51 (step S19-3). Specifically, as shown in FIG. 18, the rod member 60 is moved in the direction of the arrow B to be engaged with the hole part 51.

As shown in FIG. 19, the line member 30 is then wound around the grooves 61 and 62 in the rod member 60 (step S19-4). The rod member 60 may have the first slits 63 and 64 for connecting a plurality of grooves 61 and 62 as in the rod member 60e of FIG. 14E.

The one end part 31 and the other end part 32 of the line member 30 are then fixed in a cut formed on the rod member 60 (step S19-5). When the rod member 60 does not have a cut, this step should be omitted.

The above procedure is applied to the case in which the rod member 60 is disposed proximal to the hole member 50. When the hole member 50 is disposed proximal to the rod member 60, steps S19-1 to S19-4 should be performed in the order of S19-1, S19-4, S19-2, and S19-3.

The spacer 82 is then attached to the outer surface of the inner tubular body 71 proximally to the outer tubular body 70 and distally to the second handle 81 (step S20). This step may be omitted.

3. Method for Operating Medical Clip Device

One or more embodiments of the present invention include a method for operating the medical clip device, in which the medical clip device comprises an outer tubular body, an inner tubular body provided in the outer tubular body, a clip for holding a body tissue, that is provided at a distal side of the inner tubular body, and a line member whose one end part and the other end part extend out of the inner tubular body from an inside of the inner tubular body at a proximal side of the inner tubular body and whose a middle part is hung on the clip at the distal side of the inner tubular body, comprising the steps of: closing the clip; and moving the one end part of the line member to the proximal side. Following these steps, the operator can easily place the clip in a body and withdraw the line member from the body. When the operator needs to use other surgical device, the line member does not hinder the operator from operating the other surgical device.

One or more embodiments of the present invention also include a method for operating the medical clip device, in which the medical clip device comprises an outer tubular body, an inner tubular body provided in the outer tubular body, a clip for holding a body tissue, that is provided at a distal side of the inner tubular body, and a line member whose one end part and the other end part extend out of the inner tubular body from an inside of the inner tubular body at a proximal side of the inner tubular body and whose a middle part is hung on the clip at the distal side of the inner tubular body, comprising the steps of: closing the clip; and moving the one end part and the other end part of the line member to the proximal side. Following these steps, the operator can easily withdraw the line member together with the clip holding an exfoliated lesion from the body.

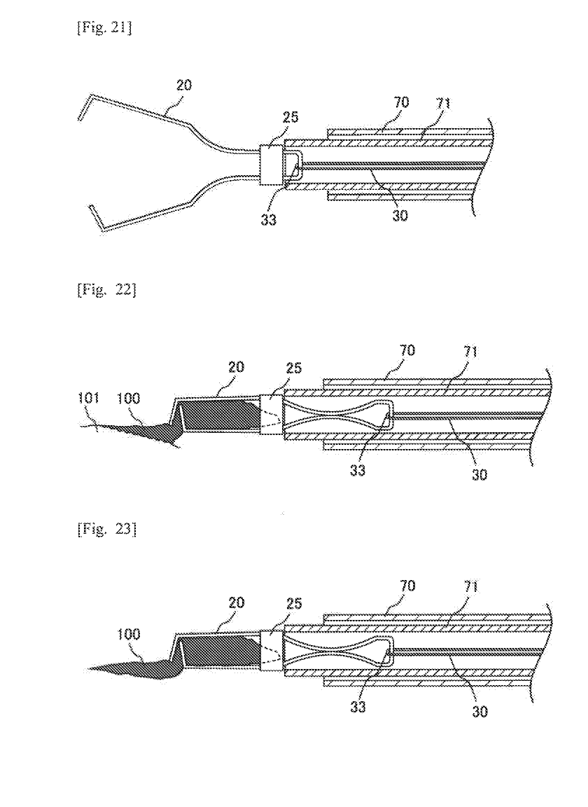

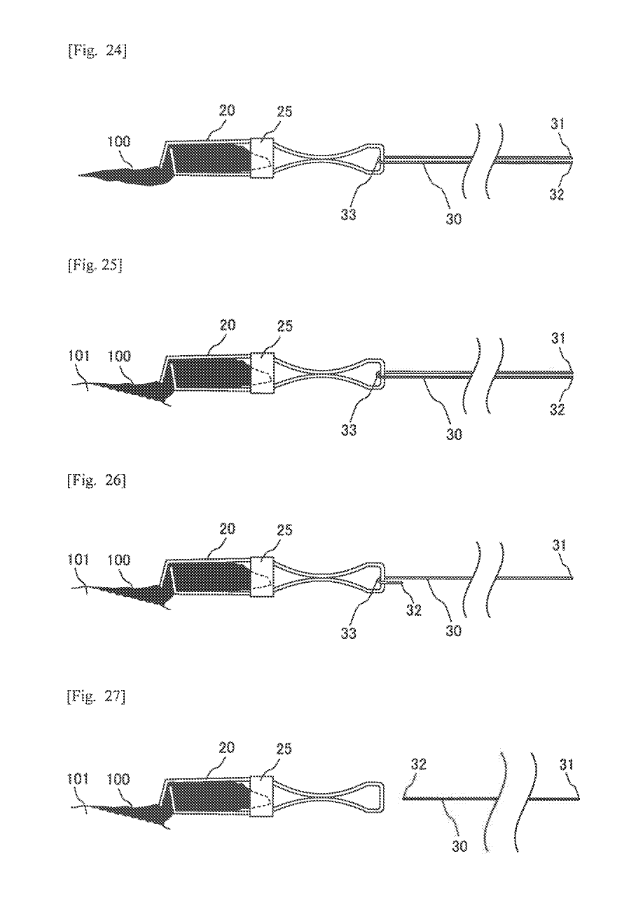

A method for operating the medical clip device 10 of one or more embodiments of the present invention will be described with reference to FIGS. 20 to 27. FIGS. 20 and 21 are side views (including partial cross-sectional views) showing the method for operating the medical clip device 10 according to one or more embodiments of the present invention, FIGS. 22 and 23 are pattern diagrams (including partial cross-sectional views) showing a method for operating the medical clip device 10 of one or more embodiments of the present invention, and FIGS. 24 to 27 are pattern diagrams showing a method for operating the medical clip device 10 of one or more embodiments of the present invention. The medical clip device 10 includes the constituent components described in the above section “1. Medical Clip Device.”

First, to make a lesion 100 noticeable, the area around the lesion 100 is sprayed with a pigment or marked. The lesion 100 is marked by cauterization with a high-frequency device, for example.

To facilitate the excision of the lesion, some physiological saline solution or hyaluronic acid is infused between the muscular layer and the submucosal layer 101 at the lesion 100 to raise the lesion 100.

The spacer 82 is then attached to the outer surface of the inner tubular body 71 proximally to the outer tubular body 70 and distally to the second handle 81 to prevent the outer tubular body 70 from accidentally moving in the axial direction when the clip device 10 is inserted into the forceps channel (step S21). This step may be omitted.

The inner tubular body 71 and the clip 20 is then placed in the outer tubular body 70 to prevent the clip 20 from scratching the forceps channel of an endoscope when the clip device 10 is inserted into the forceps channel (step S22). Specifically, the first handle 80 connected to the outer tubular body 70 is moved to the distal side with respect to the inner tubular body 71 and the second handle 81 until the outer tubular body 70 completely covers the clip 20 as shown in FIG. 20. As the outer tubular body 70 proceeds over the clip 20, the pinching base plates 21a and 21b of the clip 20 gradually get close to each other.

Optionally, the line member 30 can be anchored to the medical fixing member 40 in the following step S23. The one end part 31 and the other end part 32 of the line member 30 extending from the proximal side of the inner tubular body 71 are anchored to the medical fixing member 40 (step S23). The line member 30 is anchored to the medical fixing member 40 as described in the above section “2. Method for Producing Medical Clip Device.”

The distal side of the clip device 10 is inserted into the forceps channel of an endoscope through the forceps opening to reach the lesion 100 in a patient to be treated (step S24). The operator adjusts the position of the clip device 10 while confirming the position and condition of the lesion 100 on the images obtained from the endoscope.

The spacer 82 on the outer surface of the inner tubular body 71 is then removed from the outer tubular body 70 (step S25).

The outer tubular body 70 is then moved to the proximal side with respect to the inner tubular body 71 (step S26). Specifically, when the first handle 80 connected to the outer tubular body 70 is moved to the proximal side with respect to the second handle 81 on the inner tubular body 71, the clip 20 is gradually exposed from the outer tubular body 70 as shown in FIG. 21. As the pinching base plate 21a and the pinching base plate 21b of the clip 20, which is contracted in the outer tubular body 70, are gradually exposed from the outer tubular body 70, the clip 20 gradually opens.

The clip 20 is then closed (step S27). Specifically, after the distal side of the clip 20 is placed on the lesion 100, the one end part 31 and the other end part 32 of the line member 30 are held and moved to the proximal side. As the proximal side of the clip 20 enters the inner tubular body 71, the clip 20 gradually closes. Since the outer diameter of the fastener member 25 around the clip 20 is larger than the inner diameter of the inner tubular body 71, the fastener member 25 does not enter into the inner tubular body 71. As shown in FIG. 22, the lesion 100 is held by the clip 20. In the case in which the one end part 31 and the other end part 32 of the line member 30 are anchored to the medical fixing member 40 at step S23, the medical fixing member 40 may be held and moved to the proximal side instead of the one end part 31 and the other end part 32 of the line member 30 for closing the clip 20 to hold the lesion 100. The medical fixing member 40 enables the line member 30 to be steadily operated as well as to be easily and surely secured.

The operator can close the clip 20 by moving the one end part 31 and the other end part 32 of the line member 30 or the medical fixing member 40 with the line member 30 to the proximal side to hold the lesion 100 and then insert an electric scalpel between the lesion 100 and the submucosal layer 101 to excise the lesion 100. The operator easily excises the lesion 100 by pulling the lesion 100 with the clip 20. When excising the lesion 100, some physiological saline solution or hyaluronic acid may be infused between the muscular layer and the submucosal layer 101 at the lesion 100 to raise the lesion 100.

As shown in FIG. 23, after the excision of the lesion 100, the first handle 80 and the second handle 81 are moved to the proximal side to expose the clip 20 and the line member 30 from the outer tubular body 70 and the inner tubular body 71 (step S28). The outer tubular body 70 and the inner tubular body 71 are then withdrawn from the body. When the clip device 10 includes the medical fixing member 40 and the one end part 31 and the other end part 32 of the line member 30 are anchored to the medical fixing member 40 having an outer diameter larger than the inner diameter of the inner tubular body 71, the line member 30 should be released from the medical fixing member 40 before the withdrawal of the outer tubular body 70 and the inner tubular body 71 from the body.

As shown in FIG. 24, the one end part 31 and the other end part 32 of the line member 30 are held and moved to the proximal side (step S29). This allows the line member 30 together with the clip 20 holding the lesion 100 to be easily withdrawn from the body.

When the line member 30 is released from the medical fixing member 40 at step S27, step S29 may be replaced with the following steps S30 and S31 for taking the lesion 100 out of the body.

The one end part 31 and the other end part 32 of the line member 30 are anchored to the medical fixing member 40 again (step S30).

The medical fixing member 40 with the line member 30 is then held and moved to the proximal side (step S31). This allows the line member 30 together with the clip 20 holding the lesion 100 to be easily withdrawn from the body.

When the lesion 100 is small enough to be accommodated in the outer tubular body 71, step S28 and S29 may be replaced with a step of holding and moving the first handle 80, the second handle 81, and the one end part 31 and the other end part 32 of the line member 30 to the proximal side (step S32). This allows the outer tubular body 70, the inner tubular body 71, the clip 20, and the line member 30 to be withdrawn from the body.

When the operator needs to use other surgical device for excising the submucosal layer 101 at the lesion 100, the following steps may be taken.

As described at step S27, when the clip device 10 includes the medical fixing member 40 having an outer diameter larger than the inner diameter of the inner tubular body 71, the line member 30 is released from the medical fixing member 40 before moving the outer tubular body 70 and the inner tubular body 71 to the proximal side to withdraw them from the body. When the medical fixing member 40 with the line member 30 has an outer diameter smaller than the inner diameter of the inner tubular body 71, this step may be omitted.

As shown in FIG. 25, the first handle 80 and the second handle 81 are held and moved to the proximal side in order to move the outer tubular body 70 and the inner tubular body 71 to the proximal side (step S33). This allows the outer tubular body 70 and the inner tubular body 71 to be withdrawn from the body.

The one end part 31 of the line member 30 is held and moved to the proximal side (step S34). This allows the line member 30 to be withdrawn from the body. Specifically, as shown in FIG. 26, the other end part 31 of the line member 30 is held and pulled to the proximal side. This allows the other end part 32 of the line member 30 to be pulled to the distal side, so that the length of the line member 30 between the one end part 31 and the middle part 33 becomes longer than the length of the line member 30 between the other end part 32 and the middle part 33.

As shown in FIG. 27, pulling the one end part 31 of the line member 30 further to the proximal side allows the middle part 33 of the line member 30 to come off the clip 20, which removes the line member 30 from the clip 20. As described above, the method for operating the medical clip device 10 of one or more embodiments of the present invention enables the operator to easily place the clip 20 in a body and withdraw the line member 30 from the body. The method prevents the line member 30 from hindering the operator from smoothly conducting a surgery with other surgical device.

This application claims the benefit of the priority date of Japanese patent applications No. 2014-263290 and No. 2014-263291 filed on Dec. 25, 2014. All of the contents of the Japanese patent applications No. 2014-263290 and No. 2014-263291 filed on Dec. 25, 2014, are incorporated by reference herein.

Although the disclosure has been described with respect to only a limited number of embodiments, those skilled in the art, having benefit of this disclosure, will appreciate that various other embodiments may be devised without departing from the scope of the present invention. Accordingly, the scope of the present invention should be limited only by the attached claims.

### REFERENCE SIGNS LIST

10: a medical clip device
20: a clip
25: a fastener member
30: a line member
31: one end part (a first end part)
32: the other end part (a second end part)
33: a middle part
40: a medical fixing member
50: a hole member
51: a hole part
52: a second slit
60: a rod member
61: a groove
63 and 64: a first slit
70: an outer tubular body
71: an inner tubular body
80: a first handle
81: a second handle

1. A medical clip device comprising:
an outer tubular body;
an inner tubular body provided in the outer tubular body;
a clip provided at a distal side of the inner tubular body; and
a line member comprising a first end part, a second end part, and a middle part, wherein
the first end part and the second end part extend out of the inner tubular body from an inside of the inner tubular body at a proximal side of the inner tubular body, and the middle part is hung on the clip at the distal side of the inner tubular body.

2. The medical clip device according to claim 1, wherein
the line member is coated with a fluororesin.

3. The medical clip device according to claim 1, wherein
an inner edge part of the clip hanging the line member is chamfered.

4. The medical clip device according to claim 1, wherein
a medical fixing member is anchored to at least one of the first end part and the second end part of the line member.

5. The medical clip device according to claim 4, wherein
the medical fixing member comprises:
a hole member having a hole part; and
a rod member that is engaged with the hole part and holds the line member between the rod member and the hole member, wherein
a groove for winding the line member is formed on a surface of the rod member.

6. The medical clip device according to claim 5, wherein
a first slit is formed on the rod member, and
a plurality of the grooves are formed on the surface of the rod member and connected to each other through the first slit.

7. The medical clip device according to claim 5, wherein
a second slit is formed on an outer surface of the hole member, and
the second slit is connected to the hole part and extends along the hole part.

8. The medical clip device according to claim 5, wherein
a cut fixing a part of the line member is formed on the rod member.

9. The medical clip device according to claim 5, wherein
an elastic modulus of the hole member is higher than an elastic modulus of the rod member.

10. The medical clip device according to claim 1, further comprising
a first handle connected to a proximal side of the outer tubular body and being movable in an axial direction of the outer tubular body, and
a second handle connected to the proximal side of the inner tubular body.

11. The medical clip device according to claim 10, wherein
a projecting part fixing the line member is provided on an outer surface of the second handle.

12. The medical clip device according to claim 1, wherein
the clip comprises two pinching base plates and a fastener member provided outside of the pinching base plates, wherein
the fastener member has an outer diameter larger than an inner diameter of the inner tubular body, and is movable in the axial direction.

13. A method for producing a medical clip device, wherein the medical clip device comprises an outer tubular body, an inner tubular body provided in the outer tubular body, and a clip provided at a distal side of the inner tubular body, the method comprising:
hanging a line member on the clip, wherein the line member comprises a first end part and a second end part; and
pulling the first end part and the second end part out of the inner tubular body from an inside of the inner tubular body at a proximal side of the inner tubular body.

14. The method for producing a medical clip device according to claim 13, wherein the medical clip device further comprises a medical fixing member including a hole member having a hole part, and a rod member that is engaged with the hole part and holds the line member between the rod member and the hole member, wherein a groove for winding the line member is formed on a surface of the rod member, the method further comprising:
inserting a part of the line member into the hole part of the hole member;
holding the line member between the hole member and the rod member after engaging the rod member to the hole part; and
winding the line member around the groove of the rod member.

15. A method for operating the medical clip device according to claim 1, comprising:
closing the clip; and
moving the one end part of the line member to the proximal side.

16. A method for operating the medical clip device according to claim 1, comprising:
closing the clip; and
moving the first end part and the second end part of the line member to the proximal side.
